(19) United States
(12) Patent Application Publication
(10) Pub. No.: US 2012/0022966 A1
(43) Pub. Date: Jan. 26, 2012

(54) METHOD AND SYSTEM FOR PROVIDING A CUSTOM BIOPOLYMER TO A CUSTOMER

(75) Inventors: David RAAB, Beratzhausen (DE); Thomas Schoedl, Eichstaett (DE)

(73) Assignee: LIFE TECHNOLOGIES CORPORATION, Carlsbad, CA (US)

(21) Appl. No.: 13/194,110

(22) Filed: Jul. 29, 2011

Related U.S. Application Data
(63) Continuation of application No. 11/869,102, filed on Oct. 9, 2007.

(51) Int. Cl. G06Q 30/06

(52) U.S. Cl. 705/26.5

(57) ABSTRACT

The present invention relates to a method and a system for providing a customer biopolymer to a customer. The sequence of a customer biopolymer is transferred to a production control system of a biopolymer service provider, synthesized on a production machine of the service provider and delivered to the customer, wherein no human action is necessary to initiate or control the synthesis process.

The present invention relates to a method and a system for providing a customer biopolymer to a customer. The sequence of a customer biopolymer is transferred to a production control system of a biopolymer service provider, synthesized on a production machine of the service provider and delivered to the customer, wherein no human action is necessary to initiate or control the synthesis process.

Service providers of today are experiencing the markets growing demand for better service, speed and flexibility. This applies also to biopolymer service provider. Thus, it is important for biopolymer service provider, to be able to mix faster services with optimal flexibility.

Most biopolymer service provider already allow a customer to order customized biopolymers online. A customer connects to an Internet web site provided by a commercial biopolymer service provider, where the customer enters the sequence of the biopolymer to be ordered. Some biopolymer service providers allow their customers to edit the entered sequence by means of editors or algorithms which are integrated in the Internet web site of the service provider. A fact which adds on to the inflexibility of this applications.

In many cases it is desirable for a customer, for example, to analyze, to optimize or to edit the sequence of a biopolymer to be ordered as desired. Therefore it would be convenient to use sequence editors or sequence optimizing algorithms which are not predetermined by the Internet web site of the biopolymer service provider but can be freely chosen by the customer.

For example, web pages for in-silico handling of biopolymers and in particular of polynucleotide sequences are widely known. Such web pages provide tools e.g. for reverse translation (see, for example, R. Wernersson and A. G. Pedersen, Nucleic Acids Research 2003, Vol. 31, No. 13 3537-3539; O. R P Bininda-Edmonds, BMC Bioinformatics 2005, 6: 156-161) gene optimization (see, for example, R. Jerala and V. Turk, Nucleic Acids Research 1988, Vol. 16, No. 5 1759; G. Buell et al., Nucleic Acids Research 1985, Vol. 13, No. 6 1923; S. R. Presnell and S. A. Benner, Nucleic Acids Research 1988, Vol. 16, No. 5 1693; J.-M. Rouillard et al., Nucleic Acids Research 2004, Vol. 32, Web server issue DOI:10.1093/nar/gkh401; Harish et al., Nucleic Acids Research 2006, Vol. 34, Web server issue DOI:10.1093/nar/gkl242) and oligodesign for DNA synthesis (see, for example, D. M. Hoover and J. Lubkowksi; Nucleic Acids Research 2002, Vol. 30, No. 10 e43; C. Schretter and M. C. Milinkovitch, Bioinformatics 2006, Vol. 22, No. 1 115-116; R. Rydzanicz et al., Nucleic Acids Research 2005, Vol. 33, Web server issue W521-W525, DOI: 10.1093/nar/gki380; C. Varotto et al., Nucleic Acids Research 2001, Vol. 29, No. 21 4373-4377; S. Weckx et al., Nucleic Acids Research 2004, Vol. 32, Web server issue W170-W172, DOI: 10.1093/nar/gkh369; T. Lowe et al., Nucleic Acids Research 1990, Vol. 18, No. 7 1757).

Moreover, on the side of the biopolymer service provider there is human action needed to initiate and to monitor the synthesis and production process of the customized biopolymer ordered. On the one hand such need of human participation raises costs. On the other hand this also adds on the inflexibility of the system, as for example, synthesis can only be started if a human, e.g. a technician, is present.

Therefore it was the object of the present invention to provide a method for providing customized biopolymer to a customer which does not suffer from the drawbacks of the prior art methods, i.e. to provide a method which combines fast service with optimal flexibility.

The object of the present invention is achieved by a method for providing a customer biopolymer to a customer, comprising:
(a) allowing a customer of a biopolymer service provider to provide a biopolymer sequence via data communication through a data communication network and to define the provided biopolymer sequence or a biopolymer sequence resulting therefrom by editing as customer biopolymer sequence,
(b) transferring the customer biopolymer sequence directly into a production control system of the biopolymer service provider,
(c) synthesizing the customer biopolymer by at least one production machine of the biopolymer service provider, and
(d) delivering the customer biopolymer sequence to the customer.

A “customer biopolymer” according to the invention is selected from polynucleotides, proteins or peptides and starch. Preferably it is selected from polynucleotides, proteins or peptide.

Most preferably a customer biopolymer is a polynucleotide. The polynucleotide may be DNA, RNA or a DNA-RNA hybrid. The polynucleotide may be single-stranded or double-stranded. Preferably, the single or double-stranded polynucleotides have a length of at least 100 nucleotides, more preferably 250 nucleotides, even more preferably, at least 1,000 nucleotides and most preferably, at least 2,500 nucleotides.

According to an especially preferred embodiment, the polynucleotide being a customer biopolymer in terms of the present invention is a synthetic gene. According to the invention a synthetic gene is a DNA molecule artificially constructed using a set of oligonucleotides without requiring a physical DNA template. In a preferred embodiment the synthetic gene is a heterologous DNA molecule.

Proteins or peptides are preferably selected from any kind of enzyme or antibodies or fragments thereof showing substantially the same activity as the corresponding enzyme or antibody.

The polynucleotides, proteins or peptides may also comprises modifications as desired or selected by the customer. For example polynucleotides may be methylated on distinct nucleotides selected by the customer. Proteins or peptides may be, for example, posttranslationally modified such as by acylation, alkylation, amidation, biotinylation, formylation, glycosylation, hydroxylation, iodination, isoprenylation, lipoylation (such as prenylation, myristoylation, farnesylation, geranylation), oxidation, pegylation, phosphorylation, sulfation etc. Moreover residues like a heme moiety or nucleotides or derivatives thereof may be covalently attached (such as ADP-ribosylation, flavin attachment).

According to another embodiment of the invention it is also possible to provide a customized biopolymer being a polynucleotide which is cloned or integrated in any suitable vector. The customized polynucleotide may also be provided being transformed in any suitable cell or cell line. Suitable vectors or cell lines are known to the person skilled in the art. A particular suitable vector, cell or cell line can be selected by the customer for each customer biopolymer to be provided.

According to step (a) of the method of the present invention, the customer of a biopolymer service provider provides a biopolymer sequence via data communication through a data communication network. A “biopolymer sequence” which is provided by the customer can be a nucleotide sequence or an amino acid sequence. In case being an amino acid sequence, the biopolymer sequence is automatically backtranslated into a corresponding nucleotide sequence. It is understood, that the customer can provide more than one biopolymer sequence during step (a). If more than one biopolymer sequence is provided, each biopolymer sequence can be edited independently.

The biopolymer sequence can be provided, for example, by entering the sequence information manual via a keyboard or the like, copying from a electronic file which can be, for example, stored on a memory medium like a hard disk, an USB flash drive or a CD-ROM or downloading/copying from a database like GeneBank and transferring the respective biopolymer sequence to a suitable computerized system of the biopolymer service provider via data communication, e.g. via the Internet.

According to the invention, the term “biopolymer sequence” comprises not only the sequence itself, e.g. a polynucleotide sequence, but all forms of suitable electronic information formats or file formats which comprise the sequence information of the corresponding sequence.

“Data communication” in terms of the present invention comprises every kind of transferring or sending data known to the person skilled in art. “Data communication” may also comprise wireless data communication.

“Data communication network” means any computer network comprising a set of computers or devices connected to each other. Preferred examples of “data communication networks” are local area networks (LAN), wide area networks (WAN), in particular the Internet, and wireless networks (WLAN, WWAN). According to the invention, the Internet is the most preferred “data communication network”.

According to a preferred embodiment of the invention a customer connects preferably via his/her computer, logs on, to the biopolymer service provider on a network for data communication, like an Internet site, where he/she might have a password-protected account.

The customer enters the biopolymer sequence preferably through a web interface or another computerized interface. The use of a web interface is especially preferred. According to a preferred embodiment an interface can comprise one or more sub-interfaces, e.g. different web-based algorithms selected by the customer can be processed in different sub-interfaces or to monitor the synthesis process.

According to an other preferred embodiment, the computerized interface, preferably the web interface, does not require the installation of special software or plug-ins on a customer's computer, except a state of the art web browser. As only a state of the art browser and an Internet connection are necessary, the method according to the invention is convenient and easy to use, e.g. can be used from each todays standard personal computer.

In a preferred embodiment, the computerized interface, preferably web interface, allows for at least one of editing, saving, restoring, gene optimizing, as well as adapting the biopolymer sequence to customer desired parameters using state of the art algorithms defining the customer biopolymer sequence. “Editing” relates to each suitable manipulation of a polynucleotide sequence which is known to the person skilled in the art and which may be desired by a customer. The customer may simultaneously open several files and/or start and/or work on several different projects. According to the invention it is also possible to administer these files and/or projects via the computerized interface.

State of the art algorithms which are preferably used are selected from algorithms which are web-based and which preferably are free accessibly for each customer. Preferred algorithms relate to codon optimizing, reverse translation, oligodesign, splice-site identification and/or prediction, in-silico DNA restriction analysis, protein analysis, sequence alignment and/or comparison and sequence search. According to an especially preferred embodiment the use of all algorithms can be automated if desired by the customer. For example, sequence searches such as data bank searches may be automatized.

Web-based applications are known to the person skilled in the art. Preferred examples which could be used in the context of the invention, either on basis of existing web-based applications or by providing analogous functionalities in the computerized interface are the following:

Preferred web-based applications for codon optimizing are:

http://www.geocities.com/codonoptimizer/

http://www.evolvingcode.net/codon/sgd/index.php

http://www.changbioscience.com/primo/primoo.html

http://www.mast.queensu.ca/˜mankg/www/codonopt/codonop.html

http://www.vectorcore.pitt.edu/upgene/upgene.html

Preferred web-based applications for reverse translation are:

http://www.bioinformatics.org/sms2/rev_trans.html

http://www.vivo.colostate.edu/molkit/rtranslate/index.html

http://slam.bs.jhmi.edu/cgi-bin/gd/gdRevTrans.cgi

Preferred web-based applications for oligodesign are:

http://mcl1.ncifcrf.gov/dnaworks/dnaworks2_DNA.html

http://www.idtdna.com/analyzer/Applications/OligoAnalyzer/Default.aspx

Preferred web-based applications for splice-site identification/predictions are:

http://genes.mit.edu/GENSCAN.html

http://biogenio.com/splice/splice.cgi

Preferred web-based applications for in-silico DNA restriction are:

http://tools.neb.com/NEBcutter2/index.php

http://www.firstmarket.com/cutter/cut2.html

Preferred web-based applications for protein analysis are:

http://expasy.org/spdbv/

http://expasy.org/tools/multiident/

http://ihg.gsf.de/ihg/mitoprot.html

http://www.cbs.dtu.dk/services/NetPhos/

Preferred web-based applications for sequence alignment and/or sequence comparison and sequence search are:

http://bioinfo.hku.hk/EMBOSS/

http://www.ebi.ac.uk/clustlaw/

http://www.ch.embnet.org/software/TCoffee.html

http://130.14.29.110/BLAST/

According to an especially preferred embodiment each sequence information file format or any other file format optionally created by such a web-based application can be processed by the production control system of the biopolymer service provider.

Customer-desired parameters may be preferably selected from the group comprising codon usage, GC content and/or presence of certain sequence patterns.

“Codon usage” is the major determinant of protein translational efficiency during the elongation stage. Optimal codons may help to achieve faster translation rates and higher accuracy. In particular when using heterologous gene expression it is in many cases beneficial to optimize the nucleotide sequence to be expressed towards specific genome preferences of an organism, e.g. a fast-growing microorganisms such as Escherichia coli or Saccharomyces cerevisiae, used as a heterologous host. Methods such as the “frequency of optimal codons” (Fop) and the “codon adaptation index” (CAI) may be used to predict gene expression levels, while methods such as the “effective number of codons” (Nc) and Shannon entropy from information theory may be used to measure codon usage evenness. Moreover, for optimizing the codon usage of a customer biopolymer a query may be send to the “Codon Usage Database”.

The “GC-content” (guanine-cytosine-content) relates to the proportion of GC-based pairs in a piece of DNA. GC-pairs in the DNA are connected with three hydrogen bonds instead of two in the AT-pairs (adenine and thymine). This makes the GC-pair stronger, more resistant to denaturation by high temperatures.

“Sequence patterns” in terms of the present invention relate to portions of a DNA or protein sequence which facilitate or promote, for example, replication, transcription, translation, engineering, expression or purification of a polynucleotide sequence or an amino acid sequence. In particular, “sequence patterns” comprise any nucleotide sequence being an ORF, a promotor, a terminator, a sequence portion recognized by a restriction enzyme or codes for a protein tag which for example acts as a label or promotes protein purification or any other enzymatic activity desired and selected by a customer.

According to further embodiment of the invention, step (a) further comprises ordering of the customer biopolymer sequence via data communication through the data communication network initiating step (b).

Step (b) relates to the transfer of the customer biopolymer sequence directly into a production control system of the biopolymer service provider. Preferably, the productions control system controls all other subsequently necessary individual production steps needed to synthesize the ordered customer biopolymer.

In a preferred embodiment, the production control system can directly interact with at least one production machine without any additional human interaction.

As no human action is needed to initiate the synthesis process, it is, for example, possible for a customer to order a certain customer biopolymer even at night initiating synthesis promptly. Optionally, depending on the biopolymer ordered and the way of delivery selected, the customer can receive the biopolymer ordered the next day.

According to step (c) synthesizing of the customer biopolymer is done by at least one production machine of the biopolymer service provider. Thus use of one production machine which is able to carry out all necessary or desired synthesis steps is preferred. The production machines may use any kind of state of the art DNA or polynucleotide synthesis technologies including synthesis on basis of phosphoramidite chemistry (see, for example, EP 1 721 908; U.S. Pat. No. 6,593,088; WO 2002 050094; WO 2005 115102; WO 9525814), chip-based synthesis methods which are preferably employing light directed deprotection chemistry (US 2005 119473, US 2006 147969, U.S. Pat. No. 5,624,711, WO 2000 069553, WO 2003 235824, WO 2004 08 95 29, WO 2007 070270; WO 9500530; WO 9960156) or solid phase synthesis methods e.g. using libraries of pre-made oligonucleotides (WO 2000 075368; WO 2003 044193; WO 2007 060456; WO 9000626; WO 9947536). Of course all other DNA- or polynucleotide synthesis methods known to the person skilled in the art may be used.

According to an other preferred embodiment of the invention, synthesizing of the customer biopolymer sequence according to step (c) comprises at least one of the steps a sampling of initially synthesized oligonucleotides, cloning of a double-stranded polynucleotide product and/or expressing of such clones double-stranded polynucleotide product and purification of proteins expressed from such cloned double-stranded polynucleotide products.

Sampling of the initially synthesized polynucleotides is preferably done by means of PCR.

A double-stranded polynucleotide product can be cloned in any suitable prokaryotic or eukaryotic vector, which is known to the person skilled in the art. Moreover, prokaryotic or eukaryotic vectors which are prepared this way may be further transformed in any cell or cell line desired by the customer.

Expression of a cloned double-stranded polynucleotide product can be initiated by any kind of induction known to the person skilled in the art. Purification of such expressed proteins can be done by any suitable protein purification methods such as affinity chromatography, gel chromatography, immune purification etc.

According to another aspect of the present invention, step (c) comprises a step for controlling the quality of the end product. Preferably, this controlling is done by nucleic acid sequencing. Methods for nucleic acid sequencing such as Maxam-Gilbert sequencing or chain-termination methods, preferably dye-terminator sequencing are also known to the person skilled in the art.

Step (c) can be repeated if the quality of the end product is inadequate. In a preferred embodiment the customer can decide if the quality of the end product is inadequate and step (c) has to be repeated.

According to another embodiment, all steps of synthesizing the customer biopolymer preferably comprising the steps of DNA or polynucleotide synthesis, assembling of initially synthesized oligonucleotides, cloning of an double-stranded product and sequencing for controlling the quality of the end product are done in one integrated production machine. In a preferred embodiment all these steps are combined in a “lab on a chip”. “Lab on a chip technology is known to the person skilled in the art. Advantages of a “lab on a chip”, which can be used according to the invention are low fluid volumes consumption, higher analysis and control speed of the chip and better efficiency due to short mixing times (short diffusion distances), fast heating (short distances, high-wall surveys to fluid volume ratios, small heat capacities), better process control because of a faster response of the system (e.g. thermal control for exothermic chemical reactions), massive paralyzation due to compactness etc. Moreover, the production machine employs preferably microfluidic technology.

The evaluation of all production process steps is performed by the production control system and works without user interaction. Thus, no human action is needed to steer the production process.

However, if desired the customer can contribute to the decision process of the production control system e.g. assessing sequencing chromatograms or modifying the initial ordering parameters e.g. canceling the synthesis using a web interface.

Thus, according to another preferred embodiment of the invention, the synthesizing of the customer biopolymer can be monitored by the customer using the web interface or the computerized interface. Preferably, the customer can directly interact with the production control system. For example, the customer can stop the production process, increase the amount of ordered customer biopolymer, change the the degree of purity ordered etc.

The method according to the invention is completed by step (d): Delivering the customer biopolymer sequence to the customer. Preferably, step (d) comprises at least one of the steps preparing for shipment, billing and documentation of the ordering process.

The customer biopolymer can be provided to the customer in any suitable physical form which is desired or selected by the customer. For example, the customized biopolymer can be provided as purified polynucleotide, protein or peptide. The degree of purity, in particular in view of peptides or proteins may be selected by the customer.

The customer biopolymer can be shipped in any suitable form which is desired by the customer, e.g. in solution or solid form. Preferably it is shipped in a lyophilized form.

The customer can select the kind of package service, e.g. express package or the service company.

Another aspect of the invention is directed to a computerized system for providing a customer biopolymer to a customer, comprising:
a computerized interface for receiving a biopolymer sequence from the customer via data communication through a data communication network,
   a production control system which comprises the computerized interface or which is connected with the computerized interface, and
   at least one production machine which is directly controlled by the production control system without human action and which is adapted to synthesize a customer biopolymer sequence, the customer biopolymer sequence being the biopolymer sequence received by the computerized interface or a biopolymer sequence resulting from editing the biopolymer sequence received by the computerized interface.

According to a preferred embodiment of the computerized system, the at least one production machine is automated.

Preferably, the customer can directly interact with the production control system and/or the production machine.

### FIGURES

FIG. 1 schematically illustrates possible interactions between customer, web interface, editing, managing, customizing and ordering of biopolymer sequences, production control system, production machines, and packaging and delivering of the physical end product to the customer.

### DESCRIPTION OF THE FIGURES

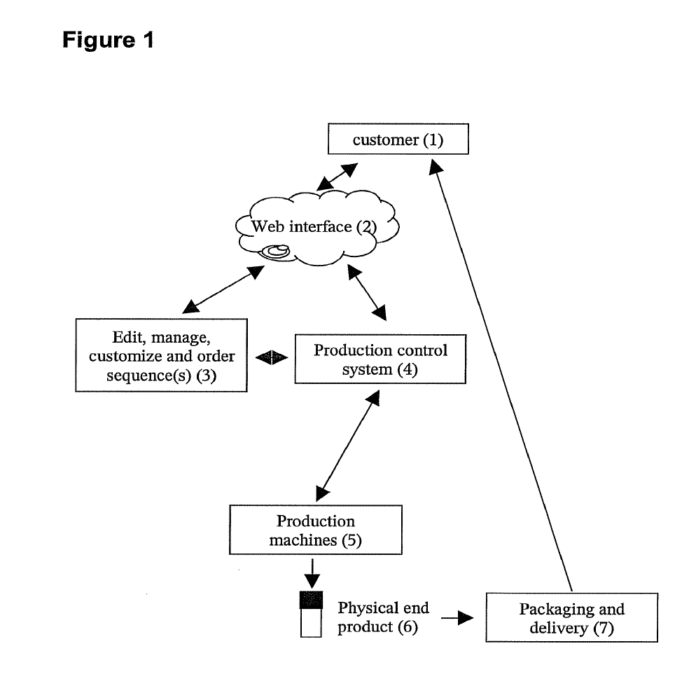

The subject-matter of the present invention will be explained in the following. Reference is made to to FIG. 1.

The scientist xy is an expert in the field of redox enzymes and has analysed the role of flavo-cytochrom-C (FCC) protein for several years. The scientist xy is therefore interested in identifying proteins having a high homology to FCC. The scientist xy has decided to turn to a biopolymer service provider of the method according to the present invention.

Thus, the scientist xy, i.e. the customer (1) connects via his computer to a biopolymer service provider on a network for data communication, i.e. the customer (1) visits the Internet site of the biopolymer service provider. The customer (1) will take all further steps via the respective web interface (2). First, the customer (1) decides to identify proteins with a high homology to FCC by sequence comparison and sequence search via a web-based application like a database search. After the respective sequences have been identified and copied to the web-interface (2) the customer (1) decides to edit these sequences (3). The customer (1) hopes to reduce the problems occurring in a heterologous expression of the proteins by codon optimization. The customer (1) thus aligns the sequences and identifies conserved amino acid positions by using web-based applications (3) (e.g. .http://bioinfo.hku.hk/EMBOSS/). Apart from a codon optimized version (amino acid sequence remains unchanged), the customer (1) edits further mutants based on the sequence alignment. The customer (1) selects also via web-interface (2) that all these constructs shall be cloned in silico in appropriate expression vectors. Due to the analysis of the 3-dimensional structure, a protein purification tag is provided by selecting appropriate restriction enzymes for the N-terminus again via the web-interface (2). After all these separate constructs are edited (3) via web interface (2), the customer (1) orders the customer biopolymer sequences also using the web interface (2). The customer polymer sequences are directly transferred to the production control system (4) of the biopolymer service provider and are then transferred in at least one production machine (5) of the biopolymer service provider. Synthesis is initiated automatically. No human interaction is necessary to initiate the synthesis process. While the synthesis takes place, the customer (1) can directly interact with the production control system (4) with regard to the ordered customer biopolymers by the web interface (2). For example, it is thus possible for the customer (1) to obtain current information on the status of the synthesis process. After the synthesis is completed, the final product (6) is packed and delivered (7) to the customer (1).

The web interface (2) may be provided by an ordinary web server computer located at the site of the biopolymer service provider or located remote therefrom. The web interface may be provided on the basis of commonly known Internet and computer technology.

If the server computer is located remote from the site of the biopolymer service provider, where the production control system with the production machine or machines are located, a data link e.g. via Internet may connect the server computer with the production control system. If the server computer is located at the site of the biopolymer service provider, where the production control system and the production machine or machines are located, there can be a fixed connection between the server computer and the production control system. Another possibility is that a common computer implements the server functionality with the web interface as well as the production control system.

Although the customer computer, e.g. an ordinary PC, can be thought of to be part of a system for providing a customer biopolymer to a customer, this computer needs no particular hardware or software functionalities. The commonly used Internet browser is sufficient, at least according to a preferred embodiment. It is within the scope of the invention that software functionalities are provided via Internet to the browser running on the customer's computer, e.g. ActiveX or Java plugins and the like. However, particular functionalities running on the local customer computer are not necessary since all necessary functionalities could be provided as functionalities of a homepage of the biopolymer service provider serving as web interface.

1. A method for providing a customer biopolymer to a customer, comprising:
(a) allowing a customer of a biopolymer service provider to provide a biopolymer sequence via data communication through a data communication network and to define the provided biopolymer sequence or a biopolymer sequence resulting therefrom by editing a customer biopolymer sequence,
(b) transferring the customer biopolymer sequence directly into a production control system of the biopolymer service provider,
(c) synthesizing the customer biopolymer by at least one production machine of the biopolymer service provider, and
(d) delivering the customer biopolymer sequence to the customer.

2. A method according to claim 1, wherein step (a) comprises entering the biopolymer sequence through a web interface or other computerized interface.

3. A method according to claim 1, wherein step (a) further comprises ordering of the customer biopolymer sequence via data communication through the data communication network by the costumer which initiates step (b).

4. A method according to claim 1, wherein the production control system directly interacts with the at least one production machine for synthesizing the customer biopolymer sequence without additional human interaction.

5. (canceled)

6. A method according to claim 1, wherein the customer biopolymer is selected from single stranded or double stranded polynucleotides sequences, proteins or peptides.

7. (canceled)

8. A method according to claim 1, wherein the biopolymer sequence provided according to step (a) is a nucleotide sequence or an amino acid sequence which is automatically back translated into a corresponding nucleotide sequence.

9. A method according to claim 2, wherein the web interface or other computerized interface allows for at least one of editing, saving, restoring, gene optimizing as well as adapting the provided biopolymer sequence to customer desired parameters using state of the art algorithms to obtain the customer biopolymer sequence.

10. (canceled)

11. A method according to claim 9, wherein the state of the art algorithms are selected from the group of algorithms relating to codon optimizing, reverse translation, oligodesign, splice site identification and/or prediction, in-silico DNA restriction analysis, protein analysis, sequence alignment and/or comparison and sequence search.

12. A method according to claim 9, wherein the customer desired parameters are selected from the group comprising codon usage, GC content and/or presence of certain sequence patterns.

13. A method according to claim 1 wherein synthesizing of the customer biopolymer sequence is done by phosphoramidite based synthesis methods, chip-based synthesis methods or solid phase synthesis methods.

14. A method according to claim 1, wherein synthesizing of the customer biopolymer sequence according to step (c) comprises at least one of the steps assembling of initially synthesized oligonucleotides, cloning of a double stranded polynucleotide product and/or expressing of such cloned double stranded polynucleotide product and purification of proteins expressed from such cloned double stranded polynucleotide product.

15. A method according to claim 1, wherein step (c) comprises a step for controlling the quality of the end product.

16. A method according to claim 15, wherein controlling the quality of the end product is done by sequencing.

17. (canceled)

18. A method according of claim 1, wherein all steps of synthesizing the customer biopolymer are integrated in one production machine.

19. (canceled)

20. A method according to claim 2, wherein the synthesizing of the customer biopolymer can be monitored by the customer using the web interface or the computerized interface.

21. A method according to claim 20, wherein the customer directly interacts with the production control system through the web interface.

22. A method according to claim 1, wherein delivering the customer biopolymer sequence to the customer comprises at least one of the steps preparing for shipment, billing and documentation of the ordering process.

23. Computerized system for providing a customer biopolymer to a customer, comprising: a computerized interface for receiving a biopolymer sequence from the customer via data communication through a data communication network, a production control system which comprises the computerized interface or which is connected with the computerized interface, and at least one production machine which is directly controlled by the production control system without human action and which is adapted to synthesize a customer biopolymer sequence, the customer biopolymer sequence being the biopolymer sequence received by the computerized interface or a biopolymer sequence resulting from editing the biopolymer sequence received by the computerized interface.

24. Computerized system according to claim 23, wherein the at least one production machine is automated.

25. Computerized system according to claim 23, wherein the production control system the production machine is configured for direct interaction with the customer through the computerized interface.
